(19) United States
(12) Patent Application Publication
(10) Pub. No.: US 2013/0045262 A1
(43) Pub. Date: Feb. 21, 2013

(54) SEDATIVE FOR EXTERNAL USE, AND SEDATIVE SHEET, AND TEXTILE, CLOTHING, WALL MATERIAL, AND HYPERTHERMIA EQUIPMENT, BRAIN VITALIZATION SHEET, BRAIN VITALIZATION SMALL PILLOW

(71) Applicant: Nobuhiro Maruyama, Miyagi (JP)

(72) Inventor: Nobuhiro Maruyama, Miyagi (JP)

(21) Appl. No.: 13/390,692

(22) PCT Filed: Oct. 29, 2010
(86) PCT No.: PCT/JP2010/069299

§ 371 Date: May 15, 2012

(30) Foreign Application Priority Data
Nov. 27, 2009 (JP) 2009-270694
Feb. 5, 2010 (JP) 2010-024847
Jun. 14, 2010 (JP) 2010-135526

(51) Int. Cl. A61M 21/02; A61M 35/00; B32B 1/00; C04B 11/00; A61K 33/06; A61K 9/14; A61P 25/20; A61F 7/08; A47C 7/74

(52) U.S. Cl. 424/402; 607/108; 607/109; 600/27; 604/289; 604/290; 428/35.6; 297/180.1; 424/696; 424/400; 106/772

(57) ABSTRACT

Disclosed are a sedative for external use, and sedative sheet, and textile, clothing, wall material, and hyperthermia equipment, brain vitalization sheet, brain vitalization small pillow which are with high productivity, adaptive for various shape and various treatment style for patients with pain, and with high treatment effect, comparing with conventional sedative for external use. A sedative for external use 10 is provided which is a bag 2 containing desert rose powder 1 prepared by pulverizing desert rose.

### TECHNICAL FIELD

The present invention is related to sedative for external use, and sedative sheet, and textile, clothing, wall material, and hyperthermia equipment to alleviate pain, stiffness in the muscle such as shoulder, cold in the patient's body, where they are contacted with, attached to, or used to cover the affected area of the patient, or the patient dresses or wears them. And also, the present invention is related to brain vitalization sheet, brain vitalization small pillow to vitalize the patient's brain.

### BACKGROUND OF THE INVENTION

### Reference Cited

### Patent Documents:

Japanese Unexamined Patent Application Publication No.2005-29555

Japanese Unexamined Patent Application Publication No.H06-261954

Previously treatment methods such as attaching plasters or wet pack sheets on the affected area, or hyperthermia are generally used for the patients with pain. In addition, a medical treatment sheet with material which emits far infrared rays is also used.

In Japanese Unexamined Patent Application Publication No.2005-29555, a treatment sheet is disclosed having material which emits gold ions at the surface area and containing material such as tourmaline which emits far infrared rays.
In Japanese Unexamined Patent Application Publication No.H06-261954, a wet pack sheet is disclosed with layers which emit far infrared rays mostly composed of ceramic layers.

### DISCLOSURE OF THE INVENTION

### Problems to be Solved by the Invention

Conventional treatment sheet disclosed in Japanese Unexamined Patent Application Publication No.2005-29555 contains expensive gold ion producing agent and has complicated material composition, which results in low productivity and high cost. And a wet pack sheet disclosed in Japanese Unexamined Patent Application Publication No.H06-261954 has sheet-like laminated composition, which results in low productivity. Also its shape is limited to sheet-like structure so that the treatment is limited to attach sheets to the patient.

The purpose of this invention is to provide sedative for external use, and sedative sheet, and textile, clothing, wall material, and hyperthermia equipment, brain vitalization sheet, brain vitalization small pillow, with high productivity, adaptive for various treatment configuration for the patients with pain, and with high sedative effect.

### BRIEF DESCRIPTION OF THE INVENTION

The invention according to claim 1 is a sedative for external use, wherein the sedative is a bag containing desert rose powder prepared by pulverizing desert rose.

The invention according to claim 2 is a sedative for external use, wherein the sedative is an ointment containing desert rose powder prepared by pulverizing desert rose.

The invention according to claim 3 is a sedative for external use according to one of claims 1 and 2, wherein the particle diameter of the desert rose powder is within the range of 10 μm to 200 μm.

The invention according to claim 4 is a sedative sheet, wherein the sedative sheet is prepared by mixing desert rose powder and silicon resin.

The invention according to claim 5 is a sedative sheet, wherein the sedative sheet is prepared by mixing desert rose powder and diatom earth.

The invention according to claim 6 is a sedative sheet according to one of claims 4 and 5, wherein the particle diameter of the desert rose powder is within the range of 10 μm to 200 μm, and the weight ratio of the desert rose powder to the sedative sheet is within the range of 40 weight % to 80 weight %.

The invention according to claim 7 is a textile, wherein the textile is prepared by the sedative sheet according to one of claims 4 and 5.

The invention according to claim 8 is clothing, wherein the clothing is prepared by the textile according to claim 7.

The invention according to claim 9 is wall material mixed with desert rose powder, wherein desert rose powder prepared by pulverizing desert rose is mixed in the wall material.

The invention according to claim 10 is a wall, wherein the wall is made of the wall material according to claim 9.

The invention according to claim 11 is a floor, wherein the floor is made of the wall material according to claim 9.

The invention according to claim 12 is a building, wherein the building is constructed using the wall according to claim 10 and the floor according to claim 11.

The invention according to claim 13 is a medical treatment sheet, wherein the sedative sheet is prepared by laminating a sheet containing desert rose powder, a plane heater, and an adhesion sheet.

The invention according to claim 14 is a hyperthermia equipment, wherein more than one medical treatment sheet is placed in the internal region of a trapezoidal housing which is formed to cover the chest, the belly, or the waist of human body, and the medical treatment sheet is formed by laminating a sheet containing desert rose powder, a plane heater, and an adhesion sheet.

The invention according to claim 15 is a hyperthermia equipment consisting of a chair and more than one medical treatment sheet placed at the backside of the chair, wherein the medical treatment sheet is formed by laminating a sheet containing desert rose powder, a plane heater, and an adhesion sheet.

The invention according to claim 16 is a hyperthermia equipment consisting of a pillow and more than one medical treatment sheet placed at the front side of the pillow, wherein the medical treatment sheet is formed by laminating a sheet containing desert rose powder, a plane heater, and an adhesion sheet.

The invention according to claim 17 is a brain vitalization sheet, wherein the brain vitalization sheet is a bag in which some Japanese papers are laminated and stored, the Japanese papers are prepared by being dried after being coated with a mixture of desert rose powder and paste dissolved in water, the desert rose powder prepared by pulverizing desert rose is mixed in the wall material.

The invention according to claim 18 is a brain vitalization small pillow, wherein the brain vitalization small pillow is a long and thin bag in which a strip of paper prepared by cutting Japanese paper dried after being coated with a mixture of desert rose powder and paste dissolved in water, the desert rose powder is prepared by pulverizing desert rose is mixed in the wall material.

The invention according to claim 19 is a method for brain vitalization, wherein the brain of a patient is vitalized by wearing a brain vitalization sheet according to claim 17 on the back, and at the same time, wearing a brain vitalization small pillow according to claim 18 on the backside of the neck.

### EFFECT OF THE INVENTION

The medical treatment can be carried out with simple treatment device set up using a sedative for external use according to claim 1 of the present invention, because the sedative can be prepared by putting desert rose powder in a bag.

The medical treatment can be carried out with simple treatment device using a sedative for external use according to claim 2 of the present invention, because the sedative is an ointment containing desert rose powder.
Textile can be formed for various shape of patients and can be applied for treatments for various affected area of patients using a textile according to claim 7.
The pain of the patients who lives in the building with wall material according to claim 9 can be alleviated.

A medical treatment sheet according to claim 13 containing desert rose powder is heated by a plane heater, so that irradiance level of far infrared ray can be increased. A hyperthermia equipment which can cover the chest, the belly, or the waist of the human body can be provided by a hyperthermia equipment according to claim 14.

Brain blood flow of prefrontal area can be increased and brain vitalization can be materialized using a brain vitalization sheet according to claim 17 and a brain vitalization small pillow according to claim 18.

According to the present invention, sedative for external use, and sedative sheet, and textile, clothing, wall material, and hyperthermia equipment, brain vitalization sheet, brain vitalization small pillow, with higher productivity, more adaptive for various treatment configuration for the patients with pain, and with higher sedative effect than the conventional sedative for external use can be provided.

### BRIEF DESCRIPTION OF THE DRAWING

FIG. 1: An external view of a sedative for external use according to the preferred embodiment 1 of the present invention.

FIG. 2: Minus ion emission data of desert rose powder.

FIG. 3: Far infrared ray emission data of desert rose powder. FIG. 3(a) is the emission intensity data, and FIG. 3(b) is the emission rate data.

FIG. 4: An external view of a sedative for external use according to the preferred embodiment 2 of the present invention.

FIG. 5: An external view of a sedative sheet according to the preferred embodiment 3 of the present invention.

FIG. 6: An external view of a sedative textile according to the preferred embodiment 4 of the present invention.

FIG. 7: An external view of a sedative clothing according to the preferred embodiment 5 of the present invention.

FIG. 8: A diagram to explain how to use a sedative for external use according to the present invention.

FIG. 9: A diagram to explain how to use a sedative sheet according to the present invention.

FIG. 10: A diagram of a room according to the preferred embodiment 8 of the present invention.

FIG. 11: A diagram of a medical treatment sheet according to the preferred embodiment 9 of the present invention. FIG. 11(a) is a perspective view, and FIG. 11(b) is a cross sectional view along the line AA.

FIG. 12: A diagram of a hyperthermia equipment according to the preferred embodiment 10 of the present invention.

FIG. 13: A diagram of a hyperthermia equipment according to the preferred embodiment 11 of the present invention.

FIG. 14: A diagram of a hyperthermia equipment according to the preferred embodiment 12 of the present invention.

FIG. 15: A diagram of a brain vitalization sheet and a brain vitalization small pillow according to the preferred embodiment 13 of the present invention. FIG. 15(a) is a diagram of a brain vitalization sheet, and FIG. 15(b) is a diagram of a brain vitalization small pillow.

FIG. 16: A diagram in which a subject sits on the desk and wears a brain vitalization sheet on the back and a brain vitalization small pillow on the backside of the neck affixed using paper tape.

FIG. 17: An image data which shows brain blood flow of prefrontal area when a subject wears a brain vitalization sheet and a brain vitalization small pillow according to the preferred embodiment 4 of the present invention. FIG. 17(a) is an image data which shows the state 15 minutes after the start of the experiment, and FIG. 17(b) is an image data which shows the state 15 minutes after the subject wears a brain vitalization sheet containing desert rose powder and a brain vitalization small pillow.

FIG. 18: A graph data which shows brain blood flow of prefrontal area when a subject wears a brain vitalization sheet and a brain vitalization small pillow according to the preferred embodiment 4 of the present invention.

FIG. 19: A comparative example using placebo (make-believe medicine), and an image data which shows brain blood flow of prefrontal area when a subject wears a placebo sheet and a placebo small pillow. FIG. 19(a) is an image data which shows the state 15 minutes after the start of the experiment, and FIG. 19(b) is an image data which shows the state 15 minutes after the subject wears a placebo sheet and a placebo small pillow.

FIG. 20: A comparative example using placebo (make-believe medicine), and an graph data which shows brain blood flow of prefrontal area when a subject wears a placebo sheet and a placebo small pillow.

### DESCRIPTION OF THE SYMBOLS

1, 4, 7, 11: desert rose powder
2: a bag
3: a sealing member
5, 8, 12: a binder
6: a case
13: an opening to let the neck through
14: a bed
10, 20: a sedative for external use
30: a sedative sheet
40: textile
50: clothing
100: a patient
101: the back of a patient
102: the shoulder of a patient
40A: a medical treatment sheet
41: a far infrared ray emission sheet
42: an adhesion member
43: a plane heater
44: a power supply cord
50A: a hyperthermia equipment
51, 52, 53: a medical treatment sheet
54: a power supply cord
140: a bed
60: a hyperthermia equipment
61: a medical treatment sheet
62: a chair
63: a power supply cord
70: a hyperthermia equipment
71, 73: a medical treatment sheet
72: a pillow
74: a power supply cord
80: a room
81, 82: wall material mixed with desert rose powder
83: a door
300: a brain vitalization sheet
500: a brain vitalization small pillow
200, 400: a bag
90A, 90B, 90C, 90D, 90E, 90F: Japanese paper dried after being coated with a mixture of desert rose powder and paste dissolved in water
91, 92, 93, 94: a strip of paper prepared by cutting Japanese paper dried after being coated with a mixture of desert rose powder and paste dissolved in water

### BEST MODE FOR CARRYING OUT THE INVENTION

### Preferred Embodiment 1

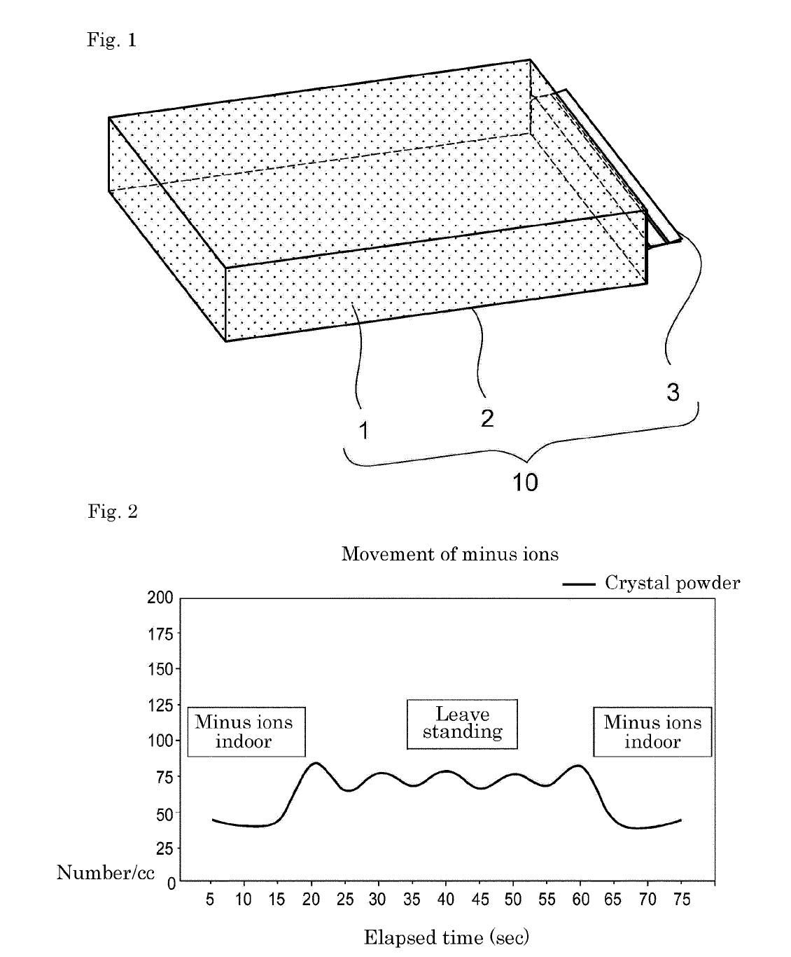

FIG. 1 is an external view of a sedative for external use according to the preferred embodiment 1 of the present invention. The sedative for external use 10 is a bag 2 containing desert rose powder 1 prepared by pulverizing desert rose. At the edge of the bag 2, a sealing member 3 is placed and affixed by, for example, thermal compression when desert rose powder is put in the bag with suitable shape. Vinyl chloride, polyethylene, etc. is used for the material of the bag 2. Other material can be used if minus ions or far infrared rays can go through it. The medical treatment to alleviate pain is carried out by placing the sedative for external use 10 in contact with the affected area of the patient. Desert rose powder used for the present invention, whose particle diameter is preferably in the range of 10 μm to 200 μm, has the function to generate minus ions and to emit far infrared rays.

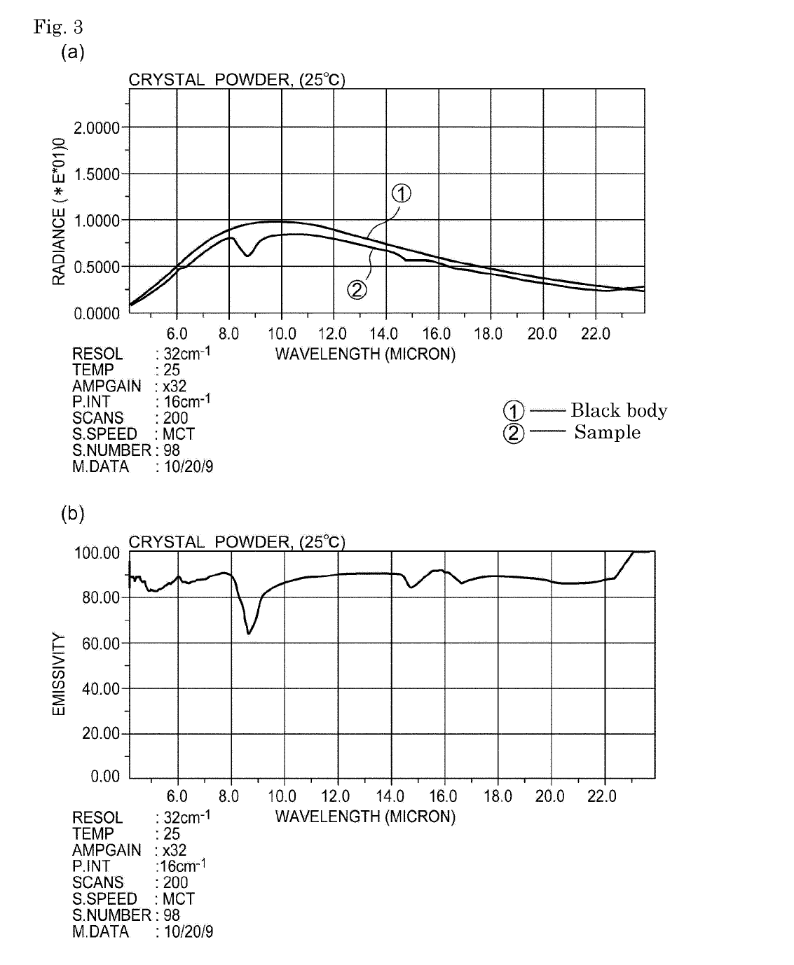

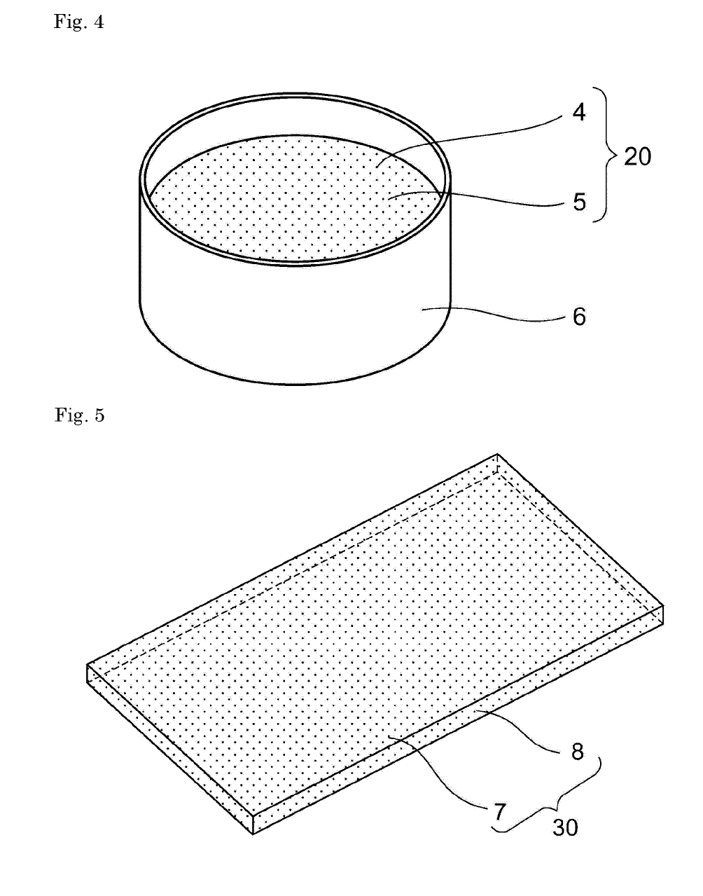

FIG. 2 is minus ion emission data of desert rose powder. And FIG. 3 is far infrared ray emission data of desert rose powder. FIG. 3(a) is the emission intensity data, and FIG. 3(b) is the emission rate data. According to FIG. 2, the average minus ion indoor density is from 65 cc⁻¹ to 84 cc⁻¹ when there is desert rose powder in a room, while it is 43 cc⁻¹ otherwise, which obviously shows that the desert rose powder is the source to generate minus ions.

FIG. 3(a) is the emission intensity data in the far infrared ray wavelength range of the desert rose powder used in the present invention. The horizontal axis represents measured wavelength (unit:μm), and the vertical axis represents emission energy (unit: W number per cm⁻²). The upper graph is the characteristics of a black body, and the lower graph is the characteristics of desert rose powder. FIG. 3(b) is the emission intensity rate of desert rose powder when the emission intensity of a black body is assumed to be 100. This data shows that desert rose powder can emit far infrared rays with high emission ratio of 60% to 90% in the wavelength range of far infrared ray.

### Preferred Embodiment 2

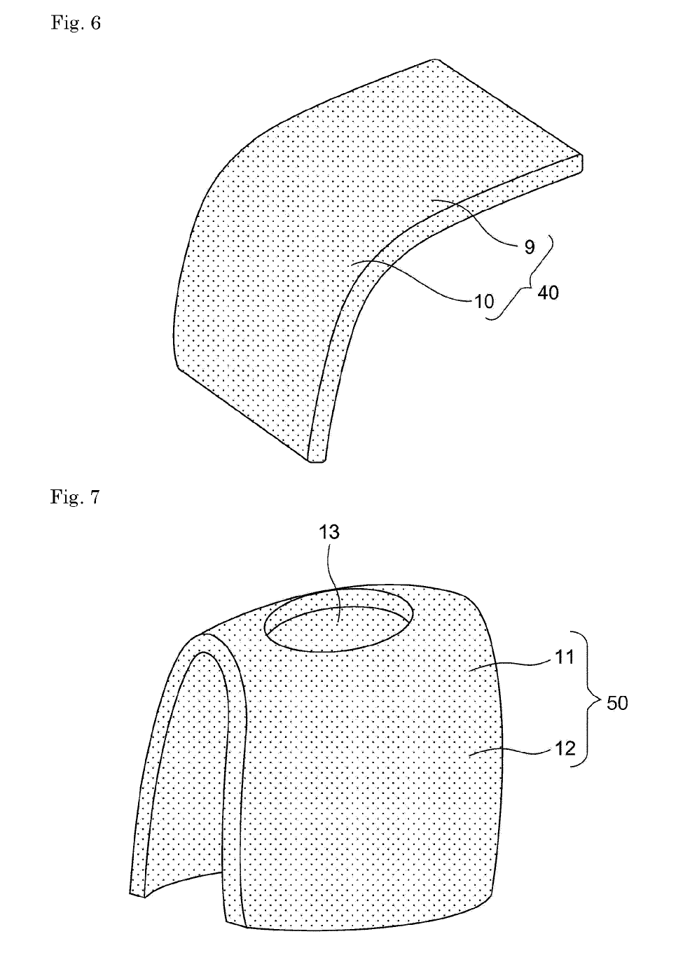

FIG. 4 is an external view of a sedative for external use according to the preferred embodiment 2 of the present invention. FIG. 4 shows a case 6 containing a sedative for external use 20. The sedative for external use 20 is an ointment where desert rose powder 4 is dispersed in binder 5. The medical treatment is carried out by applying the sedative for external use 20 as an ointment on the back, the waist, the shoulder, and the other parts of the patient with pain. Here, minus ion emission data and far infrared ray emission data of desert rose powder has been explained above using FIGS. 2 and 3. In addition, the particle diameter of the desert rose powder is in the range of 10 μm to 200 μm.

### Preferred Embodiment 3

FIG. 5 is an external view of a sedative sheet according to the preferred embodiment 3 of the present invention. The sedative for external use 30 is a sedative which is formed using desert rose powder 7 dispersed in binder 8, and extended as a sheet, and cut for appropriate size. Silicon resin, or diatom earth is used for the binder 8, but not limited to these materials.

The particle diameter of the desert rose powder is in the range of 10 μm to 200 μm, and the weight ratio of the desert rose powder to the sedative sheet is within the range of 40 weight % to 80 weight %. The medical treatment is carried out by applying the sedative sheet on the shoulder, the belly, the waist, the leg, the arm, the chest, and the back of the patient with pain. According to the applied part, the size and thickness of the sedative sheet is selected.
In addition, minus ion emission data and far infrared ray emission data of desert rose powder has been explained above using FIGS. 2 and 3, as above.

### Preferred Embodiment 4

FIG. 6 is an external view of a sedative textile according to the preferred embodiment 4 of the present invention. A textile 40 shown in FIG. 6 is composed of desert rose powder 9 and binder 10. The textile is prepared by cutting a sedative sheet formed by mixing desert rose powder and silicon resin or mixing desert rose powder and diatom earth. The shape of a textile shown in FIG. 6 is made in order to affix it around the shoulder of the patient. The medical treatment is carried out by applying the textile 40 on the shoulder, the belly, the waist, the leg, the arm, the chest, and the back of the patient with pain.

### Preferred Embodiment 5

FIG. 7 is an external view of a sedative clothing 50 according to the preferred embodiment 5 of the present invention. This clothing 50 is composed of desert rose powder 11 and binder 12 made of silicon resin or diatom earth. The clothing 50 is prepared by using the textile which is prepared from a sedative sheet formed by mixing desert rose powder and silicon resin or diatom earth.
The clothing 50 shown in FIG. 7 having an opening 13 to let the neck through is clothing preferably used as a vest. The medical treatment is carried out by putting on the upper body of the patient with pain. The shape of the clothing is not limited to the shape shown in FIG. 7. Clothing made for the lower body can also be used.

### Preferred Embodiment 6

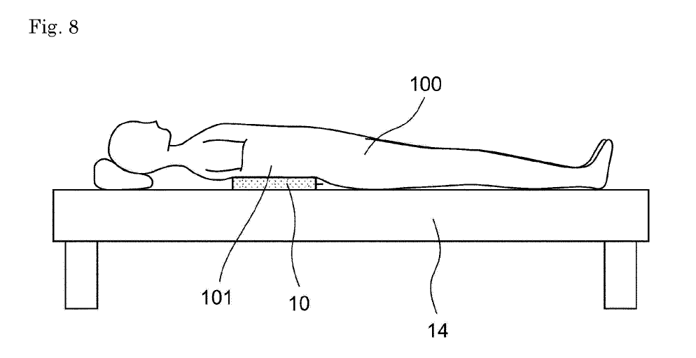

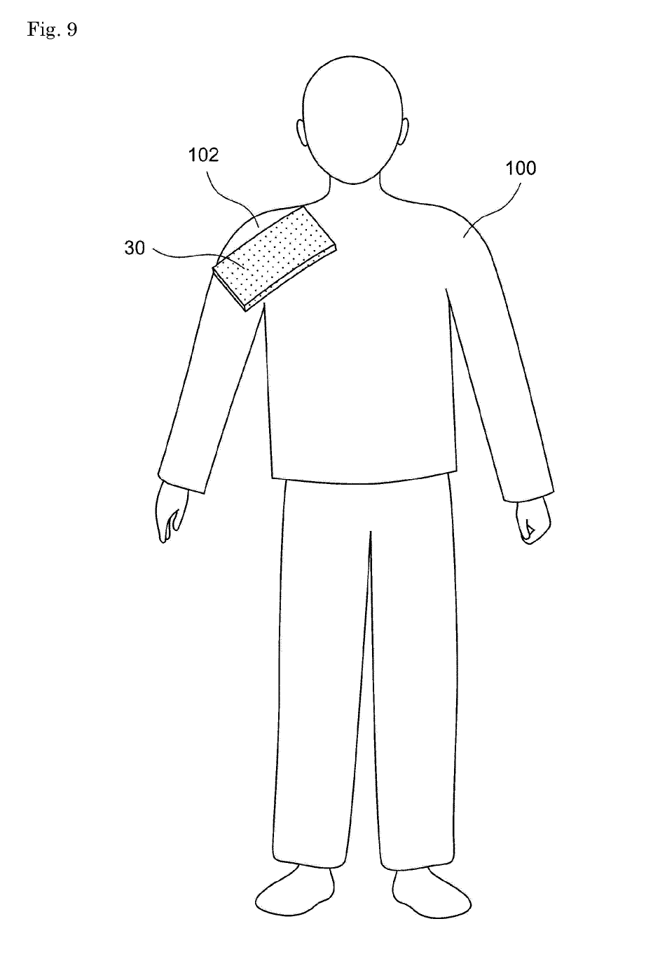

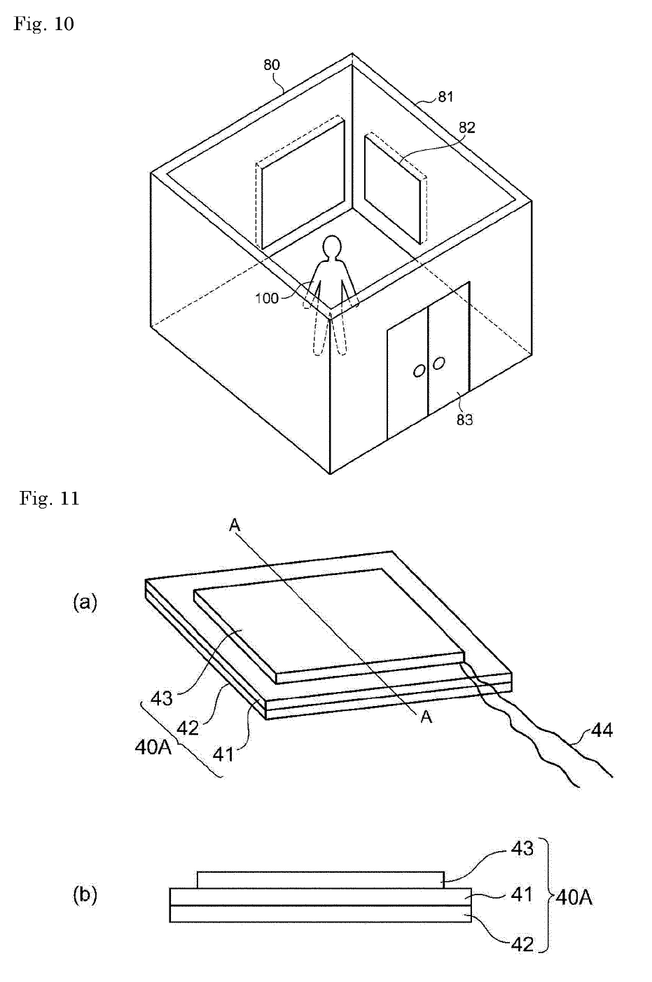

FIG. 8 is a diagram to explain how to use a sedative for external use according to the present invention. FIG. 8 shows that a patient 100 lies down on a bed 14. A sedative for external use 10 is placed under the back 101 of the patient 100. The detail about the sedative for external use 10 is explained above in the preferred embodiment 1. It is a sedative for external use where desert rose powder is packed in a bag and flattened. The medical treatment is carried out by the effect of minus ions and far infrared rays produced by the sedative for external use 10.

### Preferred Embodiment 7

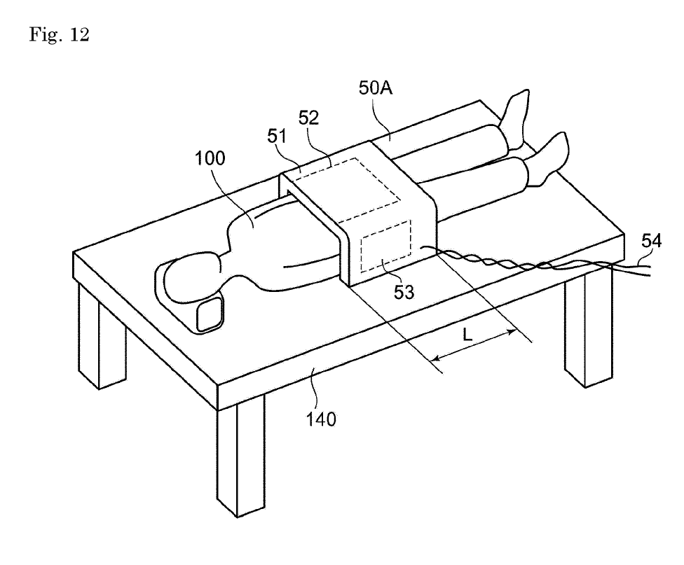

FIG. 9 is a diagram to explain how to use a sedative sheet according to the present invention. A sedative sheet 30 shown in FIG. 9 is a sedative sheet explained above for the preferred embodiment 3. This sedative sheet 30 is affixed on the shoulder 102 of the patient 100. The medical treatment is carried out on the affected area in the shoulder of the patient by the effect of minus ions and far infrared rays produced by the sedative for external use 30. In FIG. 9, the treatment for shoulder part is shown as an example. But the effect of the sedative is not limited to the shoulder, but it is effective for the desired affected area such as back, waist, chest, leg, or arm.

### Preferred Embodiment 8

Wall material can be provided in which desert rose powder made by pulverizing desert rose is mixed in concrete material or plaster board material according to wall material disclosed by claim 9. A wall or a floor can be constructed using this wall material. And also a building can be constructed using this wall material. The wall, the floor, or the building according to the preferred embodiment of the present invention will emit minus ions and far infrared rays so that the pain of the patient living inside can be alleviated. It is especially effective to alleviate his pain when the patient sits on the floor or leans back on the wall.
FIG. 10 is a diagram of a room which is constructed using wall material prepared by mixing with desert rose powder. FIG. 10 shows that at the wall area in a room 80, wall material objects 81 and 82 are installed containing desert rose powder. The pain will be alleviated by far infrared rays emitted from wall material objects 81 and 82 containing the desert rose material for the patient in the room 80.

### Preferred Embodiment 9

FIG. 11 is a diagram of a medical treatment sheet 40 according to the preferred embodiment 9 of the present invention.
FIG. 11(a) is a perspective view, and FIG. 11(b) is a cross sectional view along the line AA in FIG. 11(a).
A medical treatment sheet 40A has a structure where an adhesion member, a far infrared ray emission sheet 41, and a plane heater are stacked.
And the far infrared ray emission sheet 41 is formed by a bonded textile which is prepared by being sprayed with desert rose powder solvent and dried. But other methods (preferred embodiments) can be used to form the sheet.

A plane heater 43 is connected with a power supply cord, and produces heat by electrical power of external power supply which is not illustrated. The heat temperature is in the range of about 40° C. to 60° C., and is selected within the temperature range where human body does not suffer burn injury, and far infrared ray emission is increased from the far infrared ray emission sheet 42.
A medical treatment sheet 40A shown in FIG. 11 is in the form of a plate, but it can be manufactured in the form of other arbitrary shape such as an ellipse, a circle, or a trapezoid. Then it can be affixed on any assigned affected area. If appropriate size is selected, it can be affixed on the back of a hand or an arm.

### Preferred Embodiment 10

FIG. 12 is a diagram of corset-type hyperthermia equipment 50A according to the preferred embodiment 10 of the present invention. Corset-type hyperthermia equipment 50 with a shape of a trapezoid is placed on a bed 140, and placed to cover the chest, the belly, or the waist of the patient 100. The medical treatment can be adjusted so that it is most effective for the affected area according to the selection of size L and the position of the bed 140.
In the hyperthermia equipment 50A, medical treatment sheets 51, 52, 53 with heater which is prepared by the method according to the preferred embodiment 9 are placed inside of the plane area and both side areas of the corset-type hyperthermia equipment 50.
The temperature range of the heater can be set as higher temperature than the case according to the preferred embodiment 9, and can be more widely selected from the range of 40° C. to the maximum of 150° C. The medical treatment is carried out by emitting far infrared rays from the treatment sheets 51, 52, and 53 on the chest, the belly, or the waist of the patient 100 with pain.

### Preferred Embodiment 11

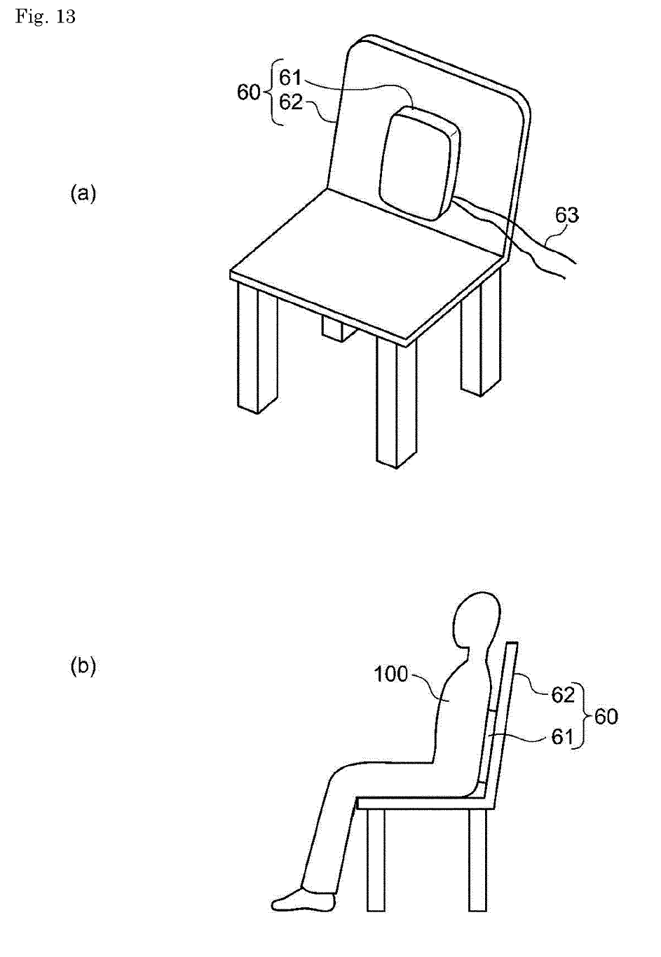

FIG. 13 is a diagram of chair-type hyperthermia equipment 60 according to the preferred embodiment 11 of the present invention. FIG. 13(a) is a perspective view, when a man sits on the chair-type hyperthermia equipment. In the hyperthermia equipment 60, a medical treatment sheet 60 with heater which is prepared by the method according to the preferred embodiment 9 is placed at the backrest of the chair 61. FIG. 13(b) is also a cross sectional view when a man sits on the chair-type hyperthermia equipment. Here, the patient 100 is applied the medical treatment for the affected range from the waist to the back of his body. In such a way according to this preferred embodiment, the patient 100 can be applied the hyperthermia treatment in his daily life.

### Preferred Embodiment 12

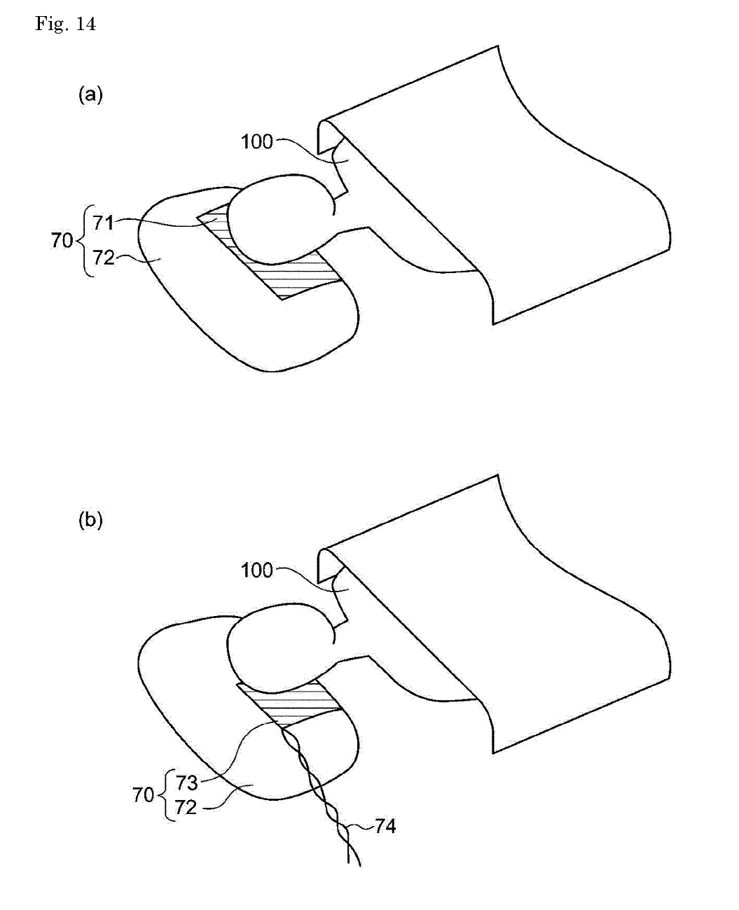

FIG. 14 is a diagram of pillow-type hyperthermia equipment 70 according to the preferred embodiment 12 of the present invention. FIG. 14(a) is hyperthermia equipment where a hyperthermia sheet 71 is affixed to a pillow 72. And FIG. 14(b) is hyperthermia equipment where a hyperthermia sheet 73 is affixed to a pillow 72. A plane heater is placed in the hyperthermia sheet 73 where electrical power is supplied via a power supply cord 74. The hyperthermia equipment 70 according to the preferred embodiment of the present invention is preferably used for the medical treatment of the affected area at the head, the cheek, and the chin of the patient 100.

### Preferred Embodiment 13

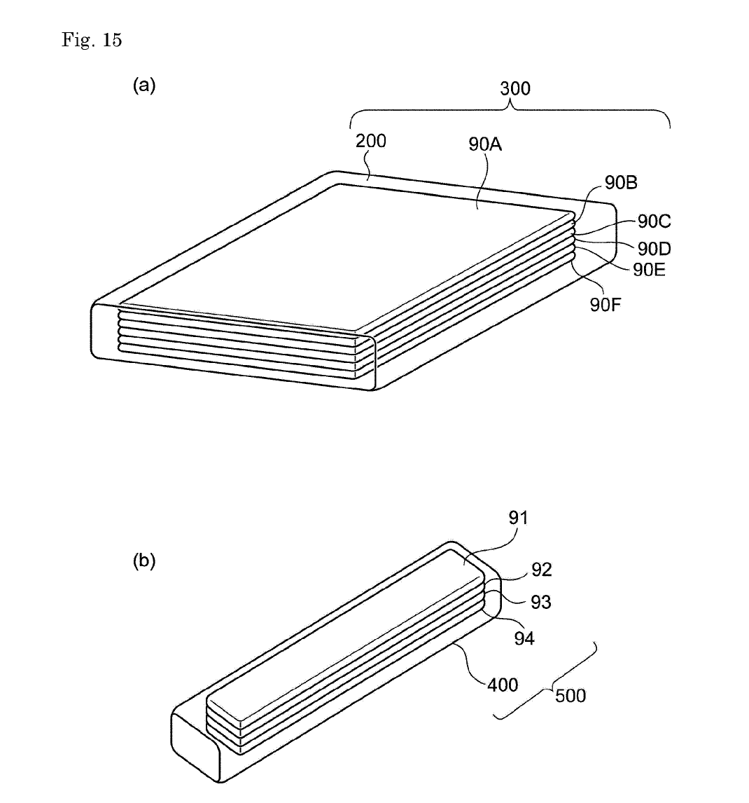

FIG. 15 is a diagram of a brain vitalization sheet 300 and a brain vitalization small pillow 500 according to the preferred embodiment 13 of the present invention. FIG. 15(a) is a brain vitalization sheet 300, and FIG. 15(b) is a brain vitalization small pillow 500. The brain vitalization sheet 300 is a sheet where Japanese papers 90A, 90B, 90C, 90D, 90E, 90F are stacked in a bag 200 after being dried after being coated with a mixture of desert rose powder and paste dissolved in water. The brain vitalization small pillow 500 is a pillow where strips of paper 91, 92, 93, 94 are put in a long and thin bag 400 prepared by cutting Japanese paper dried after being coated with a mixture of desert rose powder and paste dissolved in water.

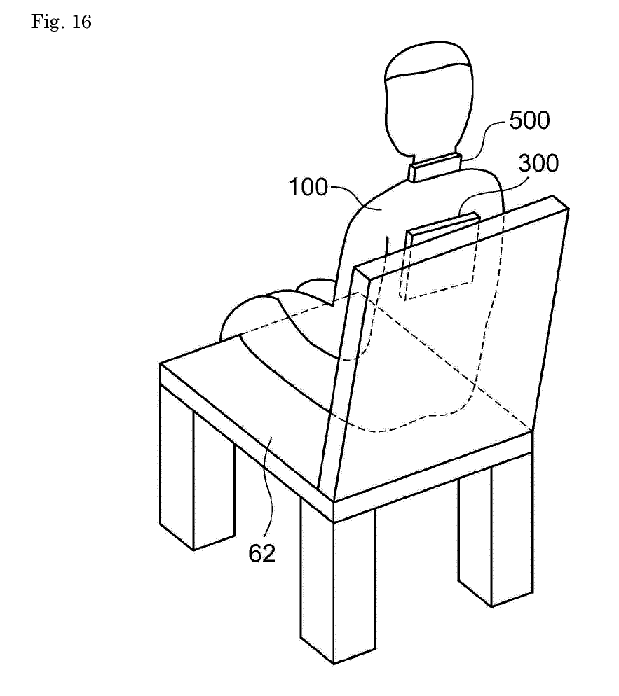

FIG. 16 is a diagram in which a subject sits on the desk and wears a brain vitalization sheet 300 on the back and a brain vitalization small pillow 500 on the backside of the neck affixed using paper tape (not illustrated).

### PRACTICAL EXAMPLE

### Practical Example 1

Table 1 is the result of the medical treatment of patients who lie in a bed using a sedative for external use which is a vinyl bag containing desert rose powder.

### (Table 1)

A treatment effect is confirmed according to Table 1 that the pain was alleviated in half for all the 20 patients among 20 patients who received the treatment.

### Practical Example 2

Table 2 is the result of the medical treatment of patients whose affected area is affixed with a sedative sheet.

### (Table 2)

A treatment effect is confirmed according to Table 2 that the pain was alleviated in half for all the 21 patients among 21 patients who received the treatment. The sedative sheet using desert rose powder is confirmed to be effective not only for muscle stiffness but also for body pain.

### Practical Example 3

Table 3 is the result of the medical treatment of patients who lie in a bed using a sedative sheet which is a ointment containing desert rose powder.

### (Table 3)

A treatment effect is confirmed according to Table 3 that the pain was alleviated in half for all the 19 patients among 21 patients who received the treatment. The sedative sheet which is an ointment using desert rose powder is confirmed to be effective not only for muscle stiffness but also for body pain.

### Practical Example 4

The brain blood flow of prefrontal area has been measured for subjects who wear a brain vitalization sheet and a brain vitalization small pillow explained in the preferred embodiment 13. The measurement has been done by using near-infrared spectroscopy (NIRS) OEG-16 manufactured by Spectratech Inc.
Four subjects (2 males, 21 years old and 23 years old and 2 females, 23 years old and 48 years old) have participated in this experiment, all of these subjects are healthy adults. And all the subjects are right-handed. The dominant hands of the subjects are unified, because the brain function differs according to the dominant hand. The experiment has been done for the subject who sit on a chair with a backrest, as explained in FIG. 16. (The procedure of the experiment)
For 20 minutes after the start of the experiment, the brain blood flow of prefrontal area has been measured under resting condition. After 20 minutes have passed, the subject wears a brain vitalization sheet on the back of the subject and a brain vitalization small pillow on the backside of the neck of the subject using adhesive paper tape. For 20 minutes after they are attached, the measurement has been continued. (The subject was instructed not to move his or her body so as not influence the brain blood flow when wearing a brain vitalization sheet and a brain vitalization small pillow.)

### (The Experiment Result)

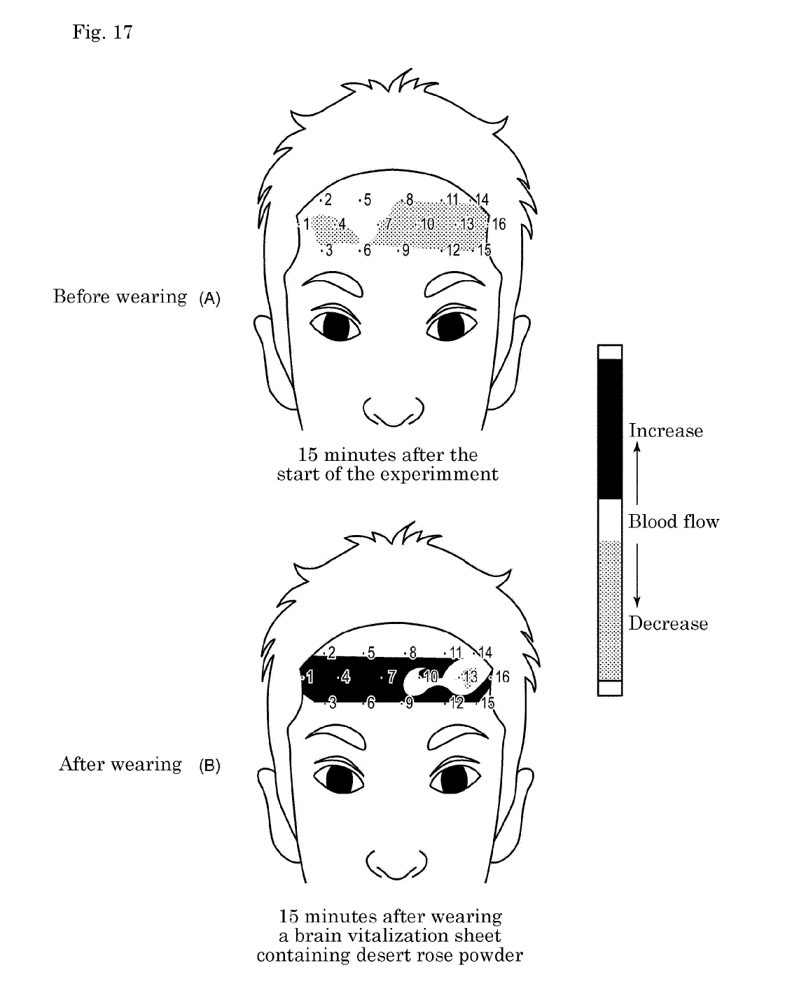

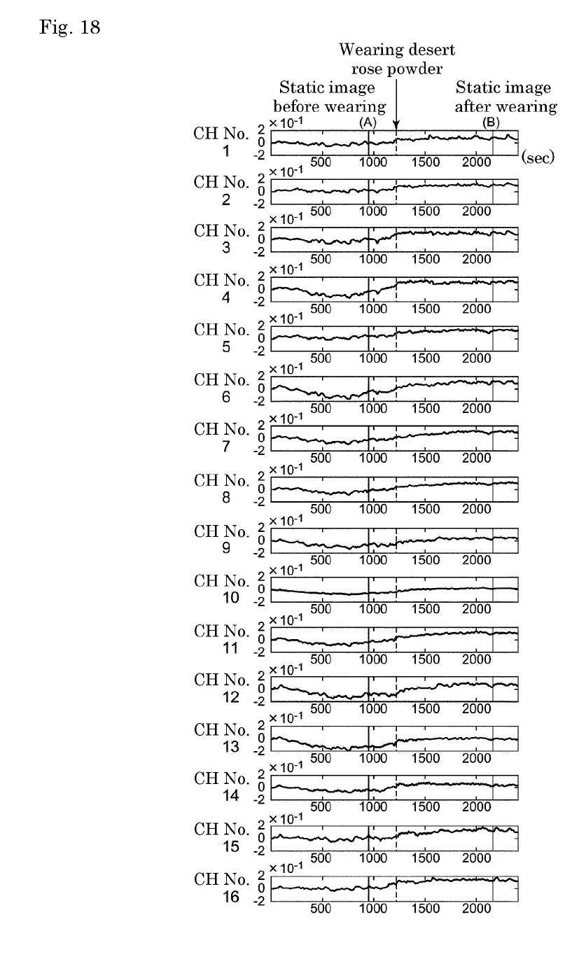

FIG. 17 is an image data which shows brain blood flow of prefrontal area when a subject wears a brain vitalization sheet and a brain vitalization small pillow according to the preferred embodiment 4 of the present invention. FIG. 17(a) is an image data which shows the state 15 minutes after the start of the experiment, and FIG. 17(b) is an image data which shows the state 15 minutes after the subject wears a brain vitalization sheet containing desert rose powder and a brain vitalization small pillow.
According to FIG. 17, the brain blood flow is small (FIG. 17(a)) before the subject wears the brain vitalization sheet and the brain vitalization small pillow. But the flow has increased over all the regions of the prefrontal area after the subject wears them.
FIG. 18 is a graph data which shows brain blood flow of prefrontal area when a subject wears a brain vitalization sheet and a brain vitalization small pillow according to the preferred embodiment 4 of the present invention. The brain blood flow has increased quantitatively over the regions (1˜16) of the prefrontal area.

### COMPARATIVE EXAMPLE

Complementary experiment was carried out according to the same procedure as the experiment of the practical example, using a placebo sheet without coating desert rose powder and a placebo small pillow (Their shape is same as shown in FIG. 15) on the different day from the experiment day of the practical example.

### (The Example Result)

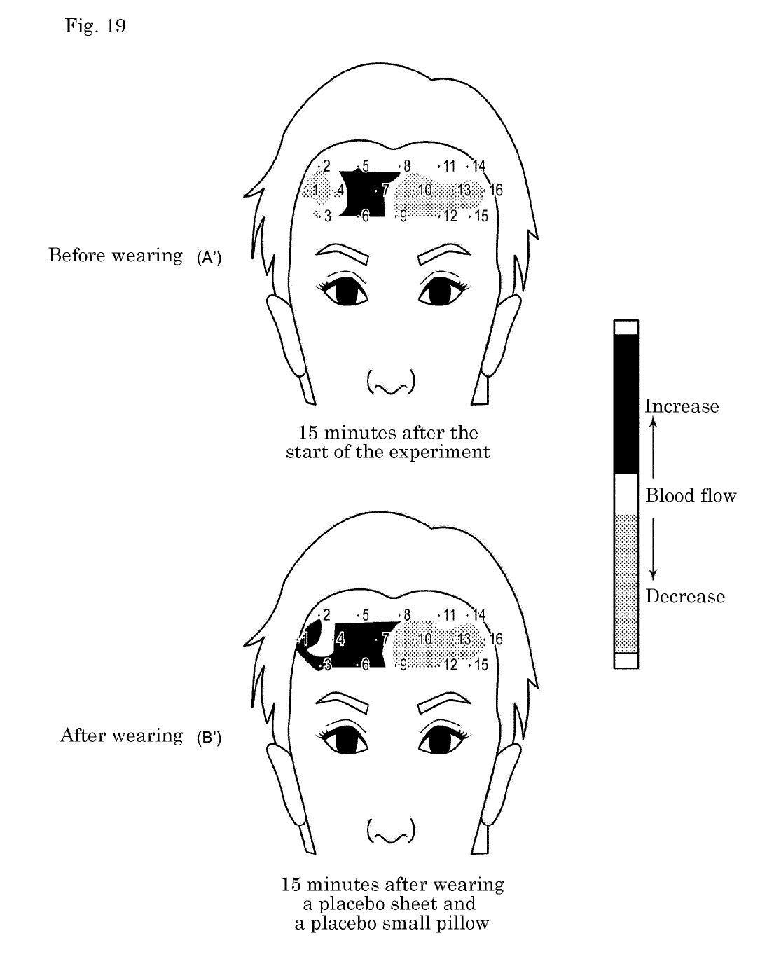

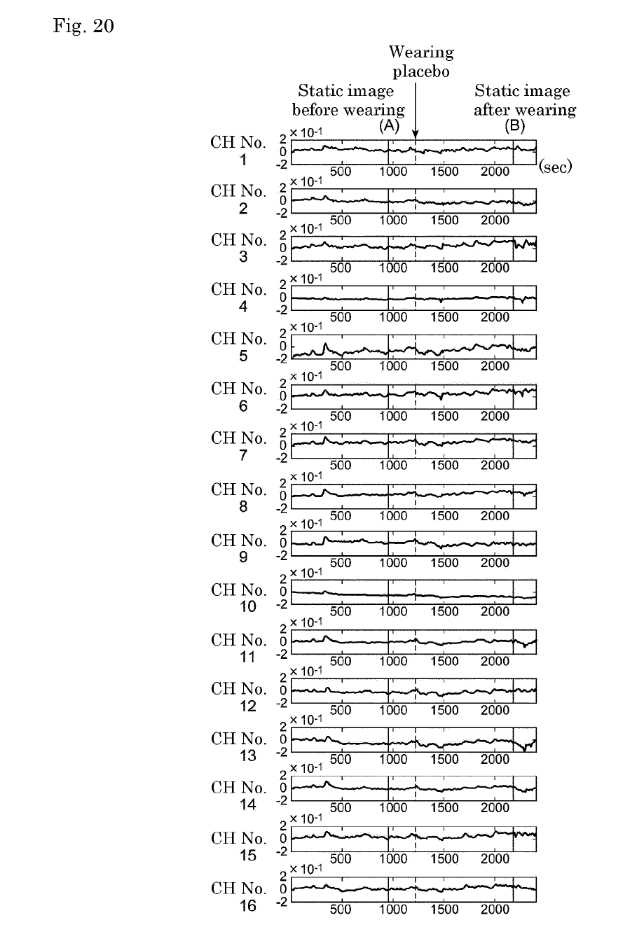

FIG. 19 is a comparative example using placebo (make-believe medicine), and an image data which shows brain blood flow of prefrontal area when a subject wears a placebo sheet and a placebo small pillow. FIG. 19(a) is an image data which shows the state 15 minutes after the start of the experiment, and FIG. 19(b) is an image data which shows the state 15 minutes after the subject wears a placebo sheet and a placebo small pillow. FIG. 19 shows that the pattern of the brain blood flow of prefrontal area does not change after wearing the placebo sheet and the placebo small pillow.
FIG. 20 is a comparative example using placebo (make-believe medicine), and a graph data which shows brain blood flow of prefrontal area when a subject wears a placebo sheet and a placebo small pillow.
FIG. 20 shows that the brain blood flow of prefrontal area does not change after wearing the placebo sheet and the placebo small pillow.

1-19. (canceled)

20. A sedative for external use, wherein the sedative is a bag containing desert rose powder prepared by pulverizing desert rose.

21. A sedative for external use, wherein the sedative is an ointment containing desert rose powder prepared by pulverizing desert rose.

22. A sedative for external use according to claim 20, wherein the particle diameter of the desert rose powder is within the range of 10 μm to 200 μm.

23. A sedative sheet, wherein the sedative sheet is prepared by mixing desert rose powder and silicon resin.

24. A sedative sheet, wherein the sedative sheet is prepared by mixing desert rose powder and diatom earth.

25. A sedative sheet according to claim 23, wherein the particle diameter of the desert rose powder is within the range of 10 μm to 200 μm, and the weight ratio of the desert rose powder to the sedative sheet is within the range of 40 weight % to 80 weight %.

26. A textile, wherein the textile is prepared by the sedative sheet according to claim 23.

27. Clothing, wherein the clothing is prepared by the textile according to claim 26.

28. Wall material mixed with desert rose powder, wherein the desert rose powder prepared by pulverizing desert rose is mixed in the wall material.

29. A wall, wherein the wall is made of the wall material according to claim 28.

30. A floor, wherein the floor is made of the wall material according to claim 28.

31. A building, wherein the building is constructed using a wall and a floor, each of the wall and the floor comprising the wall material of claim 28.

32. A medical treatment sheet, wherein the sedative sheet is prepared by laminating a sheet containing desert rose powder, a plane heater, and an adhesion sheet.

33. A hyperthermia equipment, wherein more than one medical treatment sheet is placed in the internal region of a trapezoidal housing which is formed to cover the chest, the belly, or the waist of human body, and the medical treatment sheet is formed by laminating a sheet containing desert rose powder, a plane heater, and an adhesion sheet.

34. A hyperthermia equipment consisting of a chair and more than one medical treatment sheet placed at the backside of the chair, wherein the medical treatment sheet is formed by laminating a sheet containing desert rose powder, a plane heater, and an adhesion sheet.

35. A hyperthermia equipment consisting of a pillow and more than one medical treatment sheet placed at the front side of the pillow, wherein the medical treatment sheet is formed by laminating a sheet containing desert rose powder, a plane heater, and an adhesion sheet.

36. A brain vitalization sheet, wherein the brain vitalization sheet is a bag in which some Japanese papers are laminated and stored, the Japanese papers are prepared by being dried after being coated with a mixture of desert rose powder and paste dissolved in water, the desert rose powder prepared by pulverizing desert rose is mixed in the wall material.

37. A brain vitalization small pillow, wherein the brain vitalization small pillow is a long and thin bag in which a strip of paper prepared by cutting Japanese paper dried after being coated with a mixture of desert rose powder and paste dissolved in water, the desert rose powder is prepared by pulverizing desert rose is mixed in the wall material.

38. A method for brain vitalization, wherein the brain of a patient is vitalized by wearing a brain vitalization sheet according to claim 36 on the back, and at the same time, wearing a brain vitalization small pillow on the backside of the neck, the brain vitalization pillow comprising a long and thin bag in which a strip of paper prepared by cutting Japanese paper dried after being coated with a mixture of desert rose powder and paste dissolved in water, the desert rose powder is prepared by pulverizing desert rose is mixed in the wall material.

39. A sedative for external use according to claim 21, wherein the particle diameter of the desert rose powder is within the range of 10 μm to 200 μm.
